(19) United States
(12) Patent Application Publication
(10) Pub. No.: US 2018/0171078 A1
(43) Pub. Date: Jun. 21, 2018

(54) METHOD OF MANUFACTURING GRANULAR POLYARYLENE SULFIDE, AND GRANULAR POLYARYLENE SULFIDE

(71) Applicant: Kureha Corporation, Tokyo (JP)

(72) Inventors: TAKAYUKI KIMURA, Tokyo (JP); JINYA KATAMACHI, Tokyo (JP); KENJI SUZUKI, Tokyo (JP); HIROHITO KAWAMA, Tokyo (JP)

(21) Appl. No.: 15/580,074

(22) PCT Filed: Jun. 10, 2016
(86) PCT No.: PCT/JP2016/067380

§ 371 Date: Dec. 6, 2017

(30) Foreign Application Priority Data
Jun. 12, 2015 (JP) 2015-119248

(51) Int. Cl. C08G 75/0209; C08G 75/0227; C01B 17/22; C08J 3/12

(52) U.S. Cl. CPC C08G 75/0209; C08G 75/0227; C07C 25/18; C08J 3/12; C01B 17/22

(57) ABSTRACT

To provide: a method of manufacturing granular polyarylene sulfide (PAS) with improved particle strength while improving the yield of the granular PAS by introducing and recovering PAS with a moderate molecular weight in granular PAS; and granular PAS.

A method of manufacturing granular PAS according to the present invention includes: a polymerizing step of subjecting a dihalo aromatic compound and at least one type of sulfur source selected from the group consisting of alkali metal sulfides and alkali metal hydrosulfides to polymerization reaction in an organic amide solvent; and a cooling step of cooling the reaction product mixture after the polymerizing step. Addition of a phase separation agent to the reaction product mixture is started at a point in time from the start of the polymerizing step to before the start of forming granular PAS in the cooling step, and when the temperature of the reaction product mixture is 245° C. or higher, 50 mass % or greater of the phase separation agent is added to the reaction product mixture. The polymerizing step includes a predetermined PAS prepolymer generating step, and a predetermined converting step to a high molecular weight PAS.

### TECHNICAL FIELD

The present invention relates to a method of manufacturing granular polyarylene sulfide, and granular polyarylene sulfide.

### BACKGROUND ART

Polyarylene sulfides (hereinafter, abbreviated as “PAS”) as represented by polyphenylene sulfides (hereinafter, abbreviated as “PPS”) are engineering plastics having excellent heat resistance, chemical resistance, flame retardant properties, mechanical strength, electrical properties, dimensional stability, and the like. PAS can be molded into various molded products, films, sheets, fibers, and the like by general melt processing methods such as extrusion molding, injection molding, compression molding, and the like, and therefore are widely used in a wide range of fields such as electric/electronic apparatuses, automotive apparatuses, and the like.

A method of reacting a sulfur source and dihalo aromatic compound in an organic amide solvent such as N-methyl-2-pyrrolidone or the like is known as a representative manufacturing method of PAS. An alkali metal sulfide, alkali metal hydrosulfide, or mixture thereof is generally used as the sulfur source. In a case where an alkali metal hydrosulfide is used as the sulfur source, the alkali metal hydrosulfide is used in combination with an alkali metal hydroxide.

As a method of manufacturing PAS by polymerizing a sulfur source and dihalo aromatic compound in an organic amide solvent, a method of manufacturing PAS with a high molecular weight using various polymerization auxiliary agents has been proposed. For example, Patent Document 1 discloses a method of manufacturing PAS using an alkali metal carboxylate as the polymerization auxiliary agent. Patent Document 2 discloses a method of manufacturing PPS using an alkali earth metal salt or zinc salt of an aromatic carboxylic acid as the polymerization auxiliary agent. Patent Document 3 discloses a method of manufacturing PPS using an alkali metal halide as the polymerization auxiliary agent. Patent Document 4 proposes a method of manufacturing PPS using a sodium salt of an aliphatic carboxylic acid as the polymerization auxiliary agent. Patent Document 5 discloses a method of manufacturing PAS using water as the polymerization auxiliary agent.

By adjusting the added amount and addition timing of the polymerization auxiliary agents, temperature of the polymerization reaction system, and the like, a phase separation condition where a produced polymer dense phase and produced polymer dilute phase are mixed in a liquid phase in the polymerization reaction system can be prepared. When a polymerization reaction is continued in the phase separation condition, the molecular weight of the PAS proceeds to increase, and PAS with a high molecular weight can be obtained in a granular form by gradually cooling the polymerization reaction system after the polymerization reaction. Therefore, polymerization auxiliary agents are referred to as phase separation agents.

As described in more detail, when at least one type of sulfur source selected from the group consisting of alkali metal sulfides and alkali metal hydrosulfides is reacted with a dihalo aromatic compound, a desalting condensation reaction between monomers rapidly proceeds and the conversion ratio of the dihalo aromatic compound increases. However, a polymer in this condition has low melt viscosity (molecular weight) and is in a so-called prepolymer stage. When a phase separation condition where a produced polymer dense phase and produced polymer dilute phase are mixed in a liquid phase in the polymerization reaction system is prepared in the presence of a phase separation agent, the produced polymer dense phase is dispersed in the produced polymer dilute phase by stirring, and thus a condensation reaction between prepolymers efficiently proceeds in the dense phase. As a result, the molecular weight proceeds to increase.

With granular PAS with a high molecular weight obtained by gradually cooling from a condition where the dense phase is dispersed in the dilute phase, impurities such as byproduct alkali metal salts, oligomers, and the like are easily washed and removed. Currently, granular PAS with a high molecular weight is washed by combining water washing, organic solvent washing, acid washing, and the like in a post-treatment step after polymerizing, such that PAS that does not essentially contain an alkali metal salt such as NaCl or the like can be obtained. Even in a case where sufficiently washed granular PAS with a high molecular weight is burned, ash is essentially not generated.

### CITATION LIST

### Patent Literature

Patent Document 1: JP 52-12240 B

Patent Document 2: JP 559-219332A

Patent Document 3: U.S. Pat. No. 4,038,263

Patent Document 4: JP 01-161022 A

Patent Document 5: JP 63-33775 B

### SUMMARY OF INVENTION

### Technical Problem

PAS with a moderate molecular weight of approximately 1000 to 10000 is present in a produced polymer dilute phase. Conventionally, PAS with a moderate molecular weight could not be recovered and was disposed. From the perspective of environmental problems and reducing cost of PAS, PAS with moderate molecular weight is required to be recovered as a product. Furthermore, the product yield is also required to be improved by increasing the particle strength of granular PAS and reducing crushing of particles in a post-treatment step.

In view of the foregoing, an object of the present invention is to provide: a method of manufacturing granular PAS with improved particle strength while improving the yield of the granular PAS by introducing and recovering PAS with a moderate molecular weight in granular PAS; and granular PAS.

### Solution to Problem

The present inventors discovered that the aforementioned object is achieved by starting addition of a phase separation agent to a reaction product mixture at a point in time from the start of a polymerizing step to before the start of forming granular polyarylene sulfide in a cooling step, and when the temperature of the reaction product mixture is 245° C. or higher, adding 50 mass % or greater of the phase separation agent to the reaction product mixture, when manufacturing granular PAS. Furthermore, the present inventors discovered that the aforementioned object is also achieved by reducing a specific surface area of the granular polyarylene sulfide at a point in time from the start of a polymerizing step to before the starting of forming granular polyarylene sulfide in a cooling step, when manufacturing granular PAS. Thereby, the present inventors completed the present invention.

Therefore, a first aspect of the present invention is a method of manufacturing granular polyarylene sulfide, including:
a polymerizing step of subjecting a dihalo aromatic compound and at least one type of sulfur source selected from the group consisting of alkali metal sulfides and alkali metal hydrosulfides to polymerization reaction in an organic amide solvent; and
a cooling step of cooling the reaction product mixture after the polymerizing step;
   where
addition of a phase separation agent to the reaction product mixture is started at a point in time from the start of the polymerizing step to before the start of forming granular polyarylene sulfide in the cooling step, and
when the temperature of the reaction product mixture is 245° C. or higher, 50 mass % or greater of the phase separation agent is added to the reaction product mixture;
the polymerizing step, including:
(1) a step of reacting a dihalo aromatic compound with an alkali meta sulfide at a temperature of 170 to 270° C., in an organic amide solvent containing from 0.5 to 2.4 mol of water per 1 mol of charged alkali metal sulfide, and setting the conversion ratio of the dihalo aromatic compound to 50 to 98 mol % to generate a prepolymer of a polyarlyene sulfide; and
(2) a step of adding water to a reaction system such that from 2.5 to 10 mol of water per 1 mol of charged alkali metal sulfide is in a present condition, continuing the reaction for 0.5 to 20 hours at a temperature of 245 to 290° C., increasing the conversion ratio of the dihalo aromatic compound, and converting the prepolymer to a polyarylene sulfide with a higher molecular weight.

A second aspect of the present invention is granular polyarylene sulfide with a weight average molecular weight of 60000 or less, manufactured by the method of manufacturing granular polyarylene sulfide according the first aspect of the present invention, where an amount of a material on a sieve using a screen with a 150 μm opening diameter is 86.5 mass % or greater, and the granular polyarylene sulfide has a particle strength of 88% or higher.

A third aspect of the present invention is a method of manufacturing granular polyarylene sulfide, including:
a polymerizing step of subjecting a dihalo aromatic compound and at least one type of sulfur source selected from the group consisting of alkali metal sulfides and alkali metal hydrosulfides to polymerization reaction in an organic amide solvent; and
a cooling step of cooling the reaction product mixture after the polymerizing step;
   where
a specific surface area reducing step of reducing a specific surface area of the granular polyarylene sulfide is further included at a point in time from the start of the polyermizing step to before the start of forming a granular polyarylene sulfide in the cooling step.

A fourth aspect of the present invention is granular polyarylene sulfide manufactured by the method of manufacturing granular polyarylene sulfide according to the third aspect of the present invention, where a specific surface area is 85 m²/g or less, and a particle strength is 88% or higher.

### Advantageous Effects of Invention

The present invention can provide: a method of manufacturing granular PAS with improved particle strength while improving the yield of the granular PAS by introducing and recovering PAS with a moderate molecular weight in granular PAS; and granular PAS.

### DESCRIPTION OF EMBODIMENTS

### 1. Raw Material

### 1-1. Sulfur Source

In the present invention, at least one type of a sulfur source selected from the group consisting of alkali metal sulfides and alkali metal hydrosulfides is used as the sulfur source. Examples of the alkali metal sulfide can include lithium sulfides, sodium sulfides, potassium sulfides, rubidium sulfides, cesium sulfides, mixtures of two or more thereof, and the like. Examples of the alkali metal hydrosulfide can include lithium hydrosulfide, sodium hydrosulfide, potassium hydrosulfide, rubidium hydrosulfide, cesium hydrosulfide, mixtures of two or more thereof, and the like.

The alkali metal sulfide may be used in an anhydrate, hydrate, or aqueous solution form. Of these, sodium sulfide and lithium sulfide are preferable from the perspective of being industrially available at a low cost. The alkali metal sulfide is preferably used as an aqueous solution or other aqueous mixture (in other words, a mixture with water having fluidity) from the perspective of treatment operations, weighing, and the like.

The alkali metal hydrosulfide may be used in an anhydrate, hydrate, or aqueous solution form. Of these, the sodium hydrosulfide and lithium hydrosulfide are preferable from the perspective of being industrially available at a low cost. The alkali metal hydrosulfide is preferably used as an aqueous solution or other aqueous mixture (in other words, a mixture with water having fluidity) from the perspective of treatment operations, weighing, and the like.

In general, a small amount of alkali metal hydrosulfide is produced as a byproduct in a manufacturing process of the alkali metal sulfide. A small amount of the alkali metal hydrosulfide may also be included in the alkali metal sulfide used in the present invention. In this case, the total molar amount of the alkali metal sulfide and alkali metal hydrosulfide is the added sulfur source in a charging step after a dehydrating step described later.

On the other hand, a small amount of alkali metal sulfide is produced as a byproduct in a manufacturing process of the alkali metal hydrosulfide. A small amount of the alkali metal hydrosulfide may be included in the alkali metal sulfide used in the present invention. In this case, the total molar amount of the alkali metal hydrosulfide and alkali metal sulfide is the added sulfur source in a charging step after a dehydrating step. When the alkali metal sulfide and alkali metal hydrosulfide are mixed and used, the mixture of the two will be the added sulfur source.

When the sulfur source contains the alkali metal hydrosulfide, an alkali metal hydroxide is used in combination. Examples of the alkali metal hydroxide include lithium hydroxide, sodium hydroxide, potassium hydroxide, rubidium hydroxide, cesium hydroxide, and mixtures of two or more thereof. Of these, sodium hydroxide and lithium hydroxide are preferable from the perspective of being industrially available at a low cost. The alkali metal hydroxide is preferably used as an aqueous solution or other aqueous mixture.

### 1-2. Dihalo Aromatic Compound

The dihalo aromatic compound used in the present invention is a dihalogenated aromatic compound having two halogen atoms directly bonded to an aromatic ring. Specific examples of the dihalo aromatic compounds include o-dihalobenzenes, m-dihalobenzenes, p-dihalobenzenes, dihalotoluenes, dihalonaphthalenes, methoxy-dihalobenzenes, dihalobiphenyls, dihalobenzoic acids, dihalodiphenyl ethers, dihalodiphenyl sulfones, dihalodiphenyl sulfoxides, dihalodiphenyl ketones, and the like.

Herein, halogen atoms refer to fluorine, chlorine, bromine, and iodine atoms, and in the same dihalo aromatic compound, two halogen atoms may be the same or different. These dihalo aromatic compounds can be used independently or in a combination of two or more types. p-dichlorobenzene (p-DCB) is normally frequently used.

### 1-3. Branching/Crosslinking Agent and Molecular Weight Control Agent

In order to introduce a branched or crosslinked structure into the PAS, a polyhalo compound (not necessarily an aromatic compound) where three or more halogen atoms are bonded, a halogenated aromatic compound containing an active hydrogen, a halogenated aromatic nitro compound, or the like can be used in combination. A preferable example of the polyhalo compound as a branching/crosslinking agent includes trihalobenzene.

In order to control the molecular weight or terminal group of the PAS, a monohalo organic compound can be added at an arbitrary stage in the polymerizing step. Examples of the monohalo organic compound include: monohalopropanes, monohalobutanes, monohalohexanes, aryl halides, chloroprenes, and other monohalo-substituted saturated or unsaturated aliphatic hydrocarbons; monohalocyclohexanes, monohalodecalins, and other monohalo-substituted saturated cyclic hydrocarbons; monohalobenzenes, monohalonaphthalenes, 4-chlorobenzoic acids, methyl 4-chlorobenzoates, 4-chlorodiphenyl sulfones, 4-chloronitrobenzenes, 4-chlorobenzotrifluorides, 4-chloronitrobenzenes, 4-chloroacetophenones, 4-chlorobenzophenones, benzyl chlorides, and other monohalo-substituted aromatic hydrocarbons; and the like.

Halogens atoms refer to fluorine, chlorine, bromine, and iodine. Of these halogen atoms, a chlorine atom is preferable. Furthermore, an organic compound with one substituted chlorine atom, having a substitution group such as a trifluoromethane with very low reactivity as compared to the chlorine atom is also included in the monohalo organic compounds for convenience.

### 1-4. Organic Amide Solvent

In the present invention, an organic amide solvent which is an aprotic polar organic solvent is used as a solvent for a dehydration reaction or polymerization reaction. The organic amide solvent is preferably stable with regard to alkali at a high temperature.

Specific examples of the organic amide solvent include: N,N-dimethylformamides, N,N-dimethylacetamides, and amide compounds; N-methyl-ε-caprolactams and other N-alkylcaprolactam compounds; N-methyl-2-pyrrolidones, N-cyclohexyl-2-pyrrolidones, and other N-alkylpyrrolidone compounds or N-cycloalkylpyrrolidone compounds; 1,3-dialkyl-2-imidazolidinones and other N,N-dialkylimidazolidinone compounds; tetramethyl ureas and other tetraalkyl urea compounds; hexamethylphosphoric triamides and other hexaalkylphosphoric triamide compounds; and the like. These organic amide solvents may be used independently or in a combination of two or more types.

Of these organic amide solvents, N-alkylpyrrolidone compounds, N-cycloalkylpyrrolidone compounds, N-alkylcaprolactam compounds, and N,N-dialkylimidazolidinone compounds are preferable, and N-methyl-2-pyrrolidone, N-methyl-ε-caprolactam, and 1,3-dialkyl-2-imidazolidinone are particularly preferably used. The amount of the organic amide solvent used in the polymerization reaction of the present invention is normally within a range of 0.1 to 10 kgm and preferably within a range of 0.15 to 5 kg per 1 mol of the sulfur source. When the amount of the organic amide solvent used is less than 0.1 kg, a polymerization reaction will be difficult to stably perform, and when the amount exceeds 10 kg, problems such as increased manufacturing cost and the like will occur.

### 1-5. Phase Separation Agent

In the present invention, various phase separation agents can be used in order to achieve a liquid-liquid phase separation condition. A phase separation agent refers to a compound that dissolves in an organic amide solvent alone or in the presence of a small amount of water, and reduces the solubility of PAS in the organic amide solvent. The phase separation agent itself is a compound that is not a PAS solvent.

For the phase separation agent, a conventionally known compound can generally be used as a PAS phase separation agent. Specific examples of phase separation agents include water, alkali metal carboxylates and other organic carboxylic acid metal salts, organic sulfonic acid metal salts, halogenated lithiums, alkali earth metal halides and other alkali metal halides, alkali earth metal salts of an aromatic carboxylic acid, phosphoric acid alkali metal salts, alcohols, paraffin hydrocarbons, and the like. These phase separation agents may be used independently or in a combination of two or more types. Of these, water and the organic carboxylic acid metal salts are inexpensive, which is preferable. The amount of the phase separation agent used varies based on the type of the used compound, but is generally within a range of 0.01 to 15 mol per 1 mol of the added sulfur source. The amount is preferably from 0.01 to 13 mol, more preferably from 0.02 to 12 mol, and particularly preferably from 0.03 to 10 mol. When the amount of the phase separation agent used is less than 0.01 mol, a liquid-liquid phase separation condition is difficult to achieve, and when the amount exceeds 15 mol, a polymerization reaction will not easily proceed favorably.

Of the phase separation agents, a particle modifier having a function of modifying the particle properties of the granular PAS is preferable from the perspective of particle strength of the granular PAS and the like. Specific examples of particle modifiers include water, organic carboxylic acid metal salts, organic sulfonic acid metal salts, alkali metal halides, alkali earth metal halides, alkali earth metal salts of an aromatic carboxylic acid, phosphoric acid alkali metal salts, alcohols, and paraffin hydrocarbons. Furthermore, these modifiers can be used in combination. Of these, water is particularly preferable.

### 1-6. Disulfide Compound

In the present invention, in order to obtain PAS containing a lower halogen, a polymerization reaction at a stage in at least a portion of the polymerizing step can be performed in the presence of a disulfide compound. The disulfide compound may be added at any stage in the polymerizing step. For example, in a case where the polymerizing step includes a two-stage step of a first-stage polymerizing step and second-stage polymerizing step, the compound may be added in the first-stage polymerizing step or added in the second-stage polymerizing step. Furthermore, the compound may be added at the start of the first-stage polymerizing step, in other words, in a charging step.

Furthermore, the adding period of the disulfide compound may be determined based on the conversion ratio of the dihalo aromatic compound. Specifically, the disulfide compound can be added at a point in time where the conversion ratio of the dihalo aromatic compound is from 0 to 100%, normally 45% or higher, preferably from 45 to 99.5%, more preferably from 60 to 99%, even more preferably from 70 to 98.5%, and particularly preferably from 80 to 98% in the polymerizing step, and can be present in the polymerizing step.

Examples of the disulfide compound include diphenyl disulfides (DPDS), p-p′ ditolyl disulfides, dibenzyl disulfides, dibenzoyl disulfides, dithiobenzoyl disulfides, but a diphenyl disulfide is preferable. Furthermore, a disulfide compound having a functional group can be used as all or a portion of the disulfide compounds.

The amount of the disulfide compound added when a polymerization reaction is performed in the presence of a disulfide compound in the polymerizing step is from 0.0005 to 0.015 mol, preferably from 0.0007 to 0.01 mol, more preferably from 0.0008 to 0.008 mol, even more preferably from 0.0009 to 0.006 mol, and particularly preferably from 0.001 to 0.005 mol per 1 mol of the added sulfur source. Setting the amount of the disulfide compound added within the range is important from the perspective of obtaining a granular PAS with favorable thermal stability, low gas generation during a molding process, having low halogen content and low melt viscosity, and with highly balanced performance.

The disulfide compound may be added independently in the polymerizing step or added as a mixture with an organic amide solvent.

### 2. Manufacturing Method

In an embodiment (hereinafter, referred to as “embodiment A”) of the present invention, a method of manufacturing granular PAS includes:
a polymerizing step of subjecting a dihalo aromatic compound and at least one type of sulfur source selected from the group consisting of alkali metal sulfides and alkali metal hydrosulfides to polymerization reaction in an organic amide solvent; and
a cooling step of cooling the reaction product mixture after the polymerizing step;
   where
addition of a phase separation agent to the reaction product mixture is started at a point in time from the start of the polymerizing step to before the start of forming granular PAS in the cooling step, and
when the temperature of the reaction product mixture is 245° C. or higher, 50 mass % or greater of the phase separation agent is added to the reaction product mixture;
the polymerizing step, including:
(1) a step of reacting a dihalo aromatic compound with an alkali meta sulfide at a temperature of 170 to 270° C., in an organic amide solvent containing from 0.5 to 2.4 mol of water per 1 mol of charged alkali metal sulfide, and setting the conversion ratio of the dihalo aromatic compound to 50 to 98 mol % to generate a prepolymer of a polyarlyene sulfide; and
(2) a step of adding water to a reaction system such that from 2.5 to 10 mol of water per 1 mol of charged alkali metal sulfide is in a present condition, continuing the reaction for 0.5 to 20 hours at a temperature of 245 to 290° C., increasing the conversion ratio of the dihalo aromatic compound, and converting the prepolymer to a polyarylene sulfide with a higher molecular weight. In the present specification, reaction product mixture refers to a mixture containing a reaction product generated by the aforementioned polymerization reaction, and generation starts simultaneously with the start of the aforementioned polymerization reaction.

In the present embodiment, addition of the phase separation agent to the reaction product mixture is started at a point in time from the start of a polymerizing step to before the start of forming granular PAS in the cooling step, and when the temperature of the reaction product mixture is 245° C. or higher, 50 mass % or greater of the phase separation agent is added to the reaction product mixture, and therefore, PAS with a moderate molecular weight in a melted condition has a reduced solubility in a produced polymer dilute phase and transitions to a produced polymer dense phase with a high concentration of PAS with a high molecular weight. In this condition, granular PAS is formed in the cooling step, and therefore, PAS with a moderate molecular weight can be introduced in the granular PAS and recovered as a product. As a result, in the granular PAS, the yield of material on a sieve using a screen with a 150 μm opening diameter is improved. Furthermore, with the granular PAS formed in the cooling step, specific surface area is reduced and particle strength is improved.

In the present embodiment, from the perspective of the yield and particle strength, addition of the phase separation agent to the reaction product mixture is preferably started at a point in time from after ending the polymerizing step to before the start of forming the granular polyarlyene sulfide in the cooling step.

In the present embodiment, from the perspective of the yield and particle strength, when the temperature of the reaction product mixture is 245° C. or higher, preferably 250° C. or higher, more preferably 255° C. or higher, even more preferably 260° C. or higher, and yet even more preferably 265° C. or higher, 50 mass % or greater (in other words, 50 to 100 mass %), preferably from 60 to 100 mass %, more preferably from 70 to 100 mass %, even more preferably from 80 to 100 mass %, yet even more preferably from 90 to 100 mass %, and particularly preferably from 95 to 100 mass % of the phase separation agent is added to the reaction product mixture. An upper limit of the temperature is not particularly limited, but is approximately 290° C.

In another embodiment (hereinafter, referred to as “embodiment B”) of the present invention, a method of manufacturing granular PAS includes:
a polymerizing step of subjecting a dihalo aromatic compound and at least one type of sulfur source selected from the group consisting of alkali metal sulfides and alkali metal hydrosulfides to polymerization reaction in an organic amide solvent; and
a cooling step of cooling the reaction product mixture after the polymerizing step;
   where
a specific surface area reducing step of reducing the specific surface area of granular PAS is further included at a point in time from the start of the polymerizing step to before the start of forming the granular PAS in the cooling step.

In the present embodiment, by further including the specific surface area reducing step, the particle strength of the granular PAS can be improved. For example, the specific surface area reducing step can be implemented by starting addition of the phase separation agent to the reaction product mixture at a point in time from the start of the polymerizing step to before the start of forming the granular PAS in the cooling step, and when the temperature of the reaction product mixture is 245° C. or higher, preferably 250° C. or higher, more preferably 255° C. or higher, even more preferably 260° C. or higher, and yet even more preferably 265° C. or higher, adding 50 mass % or greater (in other words, 50 to 100 mass %), preferably from 60 to 100 mass %, more preferably from 70 to 100 mass %, even more preferably from 80 to 100 mass %, yet even more preferably from 90 to 100 mass %, and particularly preferably from 95 to 100 mass % of the phase separation agent to the reaction product mixture. An upper limit of the temperature is not particularly limited, but is approximately 290° C.

In the manufacturing method according to the present invention, the weight average molecular weight of the granular PAS is preferably 60000 or less, and more preferably has a lower limit of 15000 and an upper limit of 50000, even more preferably a lower limit of 17000 and upper limit of 48000, yet even more preferably a lower limit of 18000 and upper limit of 45000, and particularly preferably a lower limit of 20000 and upper limit of 40000, from the perspective of the yield and particle strength of the granular PAS.

The manufacturing method according to the present invention may further include a dehydrating step, charging step, separating/recovering step, or the like. The steps will be described in detail below.

### 2-1. Dehydrating Step

As a preliminary step of the polymerizing step, a dehydrating step is preferably provided to adjust the coexisting water amount (also referred to as water content) in the reaction system. The dehydrating step is preferably performed by a method of heating and reacting a mixture containing an organic amide solvent and alkali metal sulfide in an inert gas atmosphere, and then discharging water to the outside of the system by distilling. In a case where alkali metal hydrosulfide is used as the sulfur source, the step is performed by heating and reacting the mixture containing alkali metal hydrosulfide and alkali metal hydroxide, and then discharging water to the outside of the system by distilling.

In the dehydrating step, dehydrating is preferably performed until the content of water including hydrated water (crystal water), aqueous medium, byproduct water, or the like is within a range of a coexisting water amount required in the charging step described later. In a case where the coexisting water amount is outside of the range required in the charging step, the lacking amount of water is preferably added.

In a case where alkali metal hydrosulfide is used as the sulfur source, in the dehydrating step, a mixture containing an organic amide solvent, alkali metal hydrosulfide, and from 0.70 to 1.07 mol, more preferably from 0.75 to 1.05 mol of an alkali metal hydroxide per 1 mol the alkali metal hydrosulfide is preferably heated and reacted, and then at least one portion of a distillate containing water is preferably discharged to the outside of a system from inside the system containing the mixture.

In a case where the molar amount of the alkali metal hydroxide per 1 mol of the alkali metal hydrosulfide added in the step is too low, the amount of sulfur components (hydrogen sulfide) volatilized in the dehydrating step increases, and abnormal reactions and PAS quality reduction is prone to occur due to reduced productivity based on a reduction in the added amount of the sulfur source, and due to increased polysulfide components in the added sulfur source remaining after dehydrating. In a case where the molar amount of the alkali metal hydroxide per 1 mol of the added alkali metal hydrosulfide is too high, degradation of the organic amide solvent may increase, a polymerization reaction may be difficult to stably perform, or PAS recovery or quality may be reduced. The molar amount of the alkali metal hydroxide per 1 mol of the alkali metal hydrosulfide added in the step is from 0.70 to 1.07 mol, and more preferably from 0.75 to 1.05 mol.

In many cases, alkali metal hydrosulfide contains a small amount of alkali metal sulfide, and the amount of the sulfur source is the total amount of the alkali metal hydrosulfide and alkali metal sulfide. It is not a problem even in a case where the alkali metal hydrosulfide, as a PAS raw material, includes an alkali metal sulfide. Furthermore, in the present invention, in a case where used with alkali metal sulfide, the molar amount with alkali metal hydroxide is calculated based on the amount (analysis value) of the alkali metal hydrosulfide, and the molar amount thereof is adjusted. Furthermore, the same also applies when alkali metal sulfide is used.

The raw materials are introduced into a reaction tank (reaction can) in the dehydrating step within a temperature range generally from ambient temperature (5 to 35° C.) to 300° C., and preferably ambient temperature to 200° C. The order of introducing the raw materials is not specified, and the raw materials may be additionally introduced during the dehydrating step. An organic amide solvent is used as the solvent used in the dehydrating step. The solvent is preferably the same as the organic amide solvent used in the polymerizing step, and N-methyl-2-pyrrolidone is particularly preferable. The amount of the organic amide solvent used is normally from 0.1 to 10 kg, and preferably from 0.15 to 5 kg per 1 mol of the sulfur source introduced in the reaction tank.

A dehydration operation is performed by heating the mixture after the introducing the raw material into the reaction tank, normally at a 300° C. or higher, and preferably within a temperature range of 100 to 250° C. and normally for 15 minutes to 24 hours, and preferably 30 minutes to 10 hours. Examples of a heating method include a method of maintaining a constant temperature, a stepwise or continuous heating method, and a method combining both of the methods. The dehydrating step may be performed by a batch method, continuous method, or a combination of both methods.

A device used to perform the dehydrating step may be the same or different than a reaction tank used in a subsequent polymerizing step. Furthermore, a material of the device is preferably a corrosion resistant material such as titanium. In the dehydrating step, a portion of the organic amide solvent is normally discharged to the outside of the reaction tank along with water. At this time, hydrogen sulfide is discharged to the outside of the system as a gas.

### 2-2. Charging Step

The charging step is a step of adjusting the amount of coexisting water with regard to the added sulfur source, the amount of dihalo aromatic compounds with regard to the added sulfur source, the amount of alkali metal hydroxides with regard to the added sulfur source, and the amount of disulfide compounds with regard to the added sulfur source, and the like, required in the polymerizing step.

“Added sulfur source” refers to a sulfur source reacted with a dihalo aromatic compound in the polymerizing step (also referred to as “active sulfur source”). In the PAS manufacturing step, a dehydrating step is generally provided, and therefore, the amount of the added sulfur source is normally calculated by an equation of [added sulfur source]=[total molar amount of added sulfur source]−[molar amount of volatilized sulfur after dehydrating].

When hydrogen sulfide is volatilized in the dehydrating step, alkali metal hydroxide is generated by an equilibrium reaction, and thus alkali metal hydroxide remains inside the system. Therefore, the amount of volatilized hydrogen sulfide needs to be accurately grasped to determine the molar amount of alkali metal hydroxide with regard to the sulfur source in the charging step. In the present invention, after the dehydrating step, alkali metal hydroxide and water can be added as necessary to the mixture remaining inside the system.

The amount of coexisting water present at the beginning of the polymerization reaction is normally within a range of 0.02 to 2.0 mol, preferably 0.05 to 1.9 mol, and more preferably 0.5 to 1.8 mol per 1 mol of the added sulfur source, in the charging step. The coexisting water amount of water can be increased during the polymerization reaction within this range.

The added amount of the dihalo aromatic compound is normally from 0.9 to 1.5 mol, preferably from 0.95 to 1.2 mol, more preferably from 1 to 1.1 mol, and particularly preferably from 1.01 to 1.08 mol per 1 mol of the added sulfur source.

The amount of the alkali metal hydroxide per 1 mol of the added sulfur source is preferably from 1.005 to 1.080 mol, more preferably from 1.010 to 1.075 mol, and particularly preferably from 1.020 to 1.073 mol. The polymerization reaction is preferably performed in a condition where the alkali metal hydroxide is in slight excess from the perspective of stably performing the polymerization reaction to obtain high quality PAS.

In the method of manufacturing PAS of the present invention, a mixture containing a less than equal molar amount of alkali metal hydroxide with regard to the sulfur source (added sulfur source) can be prepared in the charging step, such that byproducts during the polymerization reaction are suppressed from being generated or the nitrogen content derived from impurities in the generated PAS is sufficiently reduced. When the molar ratio of the alkali metal hydroxide per 1 mol of the sulfur source (added sulfur source) is 1 mol or greater (naturally includes 1.000 mol), the molar ratio of the alkali metal hydroxide per 1 mol of the sulfur source (added sulfur source) from this point is preferably within a range of 0.7 to 0.99 mol, more preferably 0.75 to 0.98 mol, and particularly preferably 0.8 to 0.97 mol. It is hypothesized that with the method of manufacturing PAS, the mixture containing less than an equal molar amount of the alkali metal hydroxide with regard to the sulfur source (added sulfur source) is prepared in the charging step to suppress a reaction between SMAB which is a byproduct and a dihalo aromatic compound such as pDCB or the like at an initial stage of the polymerizing step where the content, in other words, the amount of the dihalo aromatic compound such as pDCB or the like is high, and therefore, as a result of suppressing the generation of CPMABA which is also a byproduct, side reactions are suppressed, and thus PAS with a high purity and a high molecular weight can be obtained at a high yield.

The amount of the organic amide solvent is from 0.1 to 10 kg, and preferably from 0.15 to 5 kg per 1 mol of the sulfur source or added sulfur source.

### 2-3. Polymerizing Step

The polymerizing step is performed by heating the sulfur source and dihalo aromatic compound in the organic amide solvent. The polymerizing step may include a phase separation polymerizing step that continues a polymerization reaction in a liquid-liquid phase separation condition where a produced polymer dense phase and produced polymer dilute phase are mixed in a liquid phase in the polymerization reaction system, in the presence of a phase separation agent. In a case where the polymerizing step includes the phase separation polymerizing step, the polymerizing step may be performed in a one-stage step of only the phase separation polymerizing step, or may be performed in a two-stage step of the phase separation polymerizing step and a step without phase separation.

Furthermore, even in a case where a phase separation agent is present in a liquid phase in the polymerization reaction system after polymerizing, a liquid-liquid phase separation condition where a produced polymer dense phase and produced polymer dilute phase are mixed can be created. In other words, the phase separation agent may be added before the cooling step after the polymerization reaction.

Granular PAS can be efficiently recovered by sieving by cooling from the liquid-liquid phase separation condition.

The disulfide compound is preferably added at a 45% or higher conversion ratio of the dihalo aromatic compound. Furthermore, a polymerization auxiliary agent or other additive may be mixed before the polymerizing step or during the polymerizing step.

The polymerization reaction is generally performed at a range of 170° C. or higher (for example, 170 to 290° C.), but from the perspective of yield and particle strength of the granular PAS, the polymerization temperature is preferably 250° C. or higher (for example, 250 to 290° C.). Furthermore, the polymerization reaction is preferably performed in a two-stage step of a first-stage polymerizing step and second-stage polymerizing step. A method of maintaining a constant temperature, a stepwise or continuous heating method, or a combination of both methods is used as the heating method. The polymerization reaction time is generally within a range of 10 minutes to 72 hours, and preferably 30 minutes to 48 hours. The organic amide solvent used in the polymerizing step is normally from 0.1 to 10 kg, and preferably from 0.15 to 5 kg per 1 mol of the added sulfur source. The amount thereof may be changed during the polymerization reaction within this reaction.

A method is preferable where a liquid phase in the polymerization reaction system is converted to a phase separation condition at a stage where the conversion ratio of dihalo aromatic compound reaches 80 to 99 mol % after starting a polymerization reaction, and the polymerization reaction is continued. When a liquid phase in a high temperature condition is converted to a phase separation condition, a phase separation agent is preferably added, or the amount of an additive functioning as a phase separation agent is preferably increased. The phase separation agent is not particularly limited, but water is preferably from the perspective of low cost and easy control of the polymerization reaction and post-treatment.

In the manufacturing method of the present invention, the polymerization reaction may be performed in the presence of a disulfide compound, but the polymerizing step is preferably performed by at least the first-stage polymerizing step and second-stage polymerizing step.

Polymerization is performed within a temperature range of 170 to 270° C., and preferably 180 to 265° C. When the polymerization temperature is too low, the polymerization rate is too low, and conversely, when the temperature exceeds 270° C., generated PAS and the organic amide solvent are more likely to decompose, and the degree of polymerization of the generated PAS is very low.

### 2-3-1. First-Stage Polymerizing Step

The first-stage polymerizing step is a stage where the conversion ratio of the dihalo aromatic compound reaches 80 to 99%, preferably 85 to 98%, and more preferably 90 to 97% after starting a polymerization reaction, and is a step before the liquid phase achieves a phase separation condition. The conversion ratio of the dihalo aromatic compound is a value determined based on gas chromatography of the amount of dihalo aromatic compounds remaining in a reaction mixture, and calculated from equations below based on the residual amount thereof, added amount of the dihalo aromatic compounds, and added amount of the sulfur source.

In a case where the dihalo aromatic compound (abbreviated as “DHA”) is added at a molar amount in excess as compared to the sulfur source, the conversion ratio is calculated based on an equation below:

Conversion ratio=[(DHA added amount (mol)−DHA residual amount (mol)]/[DHA added amount (mol)−DHA excess amount (mol)]×100.

In other cases, the conversion ratio is calculated based on an equation below:

Conversion ratio=[(DHA added amount (mol)−DHA residual amount (mol)]/[DHA added amount (mol))]×100.

In the aforementioned manufacturing method, a polymer (also referred to as a “prepolymer”) with a melt viscosity measured at a temperature of 310° C. and shear rate of 1200 sec⁻¹ that is normally from 0.5 to 30 Pa·s is preferably generated in the first-stage polymerizing step.

In the first-stage polymerizing step, a phase separation condition does not occur. In the second-stage polymerizing step adding the phase separation agent, the liquid phase in the polymerization reaction system is phase separated into a polymer dense phase with a high content of polymer (prepolymer) generated by the first-stage polymerization and a polymer dilute phase with a low content of the polymer. The phase separation condition can be clearly visually observed.

### 2-3-2. Second-Stage Polymerizing Step

In a case where water is used as the phase separation agent in the second-stage polymerizing step, the coexisting water amount in the reaction system in the second-stage polymerizing step is preferably adjust within a range that is normally from 2 to 5 mol, preferably from 2.1 to 4.5 mol, more preferably from 2.2 to 4 mol, and particularly preferably from 2.3 to 3.5 mol. When the coexisting water amount in the reaction system is less than 2 mol or exceeds 5 mol, the degree of polymerization of the generated PAS is reduced.

In the second-stage polymerizing step, in a case where a phase separation agent other than water is used as the phase separation agent (at least one type of phase separation agent selected from the group consisting of organic carboxylic acid metal salts, organic sulfonic acid metal salts, alkali metal halides, alkali earth metal halides, alkali earth metal salts of an aromatic carboxylic acid, phosphoric acid alkali metal salts, alcohols, and paraffin hydrocarbons), the phase separation agent is preferably present at a range of 0.01 to 3 mol, preferably 0.02 to 2 mol, more preferably 0.03 to 1 mol, and particularly preferably 0.04 to 0.5 mol.

Water and a phase separation agent other than water can be used in combination as the phase separation agent. In this aspect, the coexisting water amount in the reaction system is preferably within a range that is normally from 0.01 to 7 mol, preferably from 0.1 to 4 mol, and more preferably from 1 to 3.5 mol per 1 mol of the added sulfur source, and the phase separation agent other than water is preferably within a range that is normally from 0.01 to 3 mol, preferably from 0.02 to 1 mol, and more preferably from 0.03 to 0.5 mol per 1 mol of the added sulfur source. Strict adjustment of the ratio of phase separation agent/added sulfur charge leads to a reduction of a low molecular weight material or oligomer.

The polymerization temperature in the second-stage polymerizing step is within a range of 240 to 290° C. When the polymerization temperature in the second-stage polymerizing step is lower than 240° C., PAS with an adjusted melt viscosity is difficult to obtained, and when the temperature exceeds 290° C., the generated PAS or organic amide solvent may decompose. Furthermore, a temperature of 245 to 280° C., and particularly 250 to 275° C. is preferable, because PAS with an adjusted melt viscosity will be easy to obtain. The second-stage polymerizing step in the present invention is not a step of simply separating/granulating a PAS prepolymer generated in the first-stage polymerizing step, but is a step for inducing increase in the degree of polymerization of the PAS prepolymer.

In the second-stage polymerizing step, a polymerization reaction is continued in a phase separation condition where a produced polymer dense phase and produced polymer dilute phase are mixed in a liquid phase in the polymerization reaction system, in the presence of a phase separation agent. The PAS concentration of the dense phase is normally from 30 to 70 mass %, preferably from 40 to 60 mass %, and more preferably from 45 to 55 mass %. The PAS concentration of the dilute phase is normally from 0.1 to 15 mass %, preferably from 0.5 to 10 mass %, and more preferably from 1 to 8 mass %.

When a liquid-liquid phase separation condition where a produced polymer dense phase and produced polymer dilute phase are mixed in a liquid phase in the polymerization reaction system is prepared in the presence of a phase separation agent, the produced polymer dense phase is dispersed in the produced polymer dilute phase by stirring, and thus a condensation reaction between prepolymers efficiently proceeds in the dense phase.

The polymerization reaction method may be a batch method, continuous method, or a combination of both methods. With batch polymerization, a method using two or more reaction tanks can be used as desired in order to reduce the polymerization cycle time.

In particular, in embodiment A, the polymerizing step includes:
(1) a step of reacting an alkali metal sulfide and dihalo aromatic compound at a temperature of 170 to 270° C., preferably 200 to 265° C., and more preferably 250 to 262° C. in an organic amide solvent containing from 0.5 to 2.4 mol, preferably from 1.0 to 2.0 mol, and more preferably from 1.3 to 1.7 mol per 1 mol of charged alkali metal sulfide, and setting the conversion ratio of the dihalo aromatic compound to 50 to 98 mol %, preferably 70 to 96 mol %, and more preferably 90 to 94 mol % to generate a polyarylene sulfide prepolymer; and
(2) a step of adding water to the reaction system so as to achieve a condition where from 2.5 to 10 mol, preferably from 2.5 to 5.0 mol, and more preferably from 2.6 to 3.0 mol of water is present per 1 mol of the charged alkali metal sulfide, continuing the reaction at temperature of 245 to 290° C., preferably 250 to 280° C., and more preferably 260 to 270° C. for 0.5 to 20 hours, preferably 1.0 to 10 hours, and more preferably 1.5 to 3.0 hours, increasing the conversion ratio of the dihalo aromatic compound, and converting the prepolymer to a polyarylene sulfide with a high molecular weight.

### 2-4. Cooling Step

The present invention includes a cooling step of cooling the reaction product mixture after the polymerizing step.

In the cooling step, the liquid phase containing the generated polymer is cooled. In the cooling step, the liquid phase is preferably gradually cooled and not rapidly cooled by solvent flushing or the like, because a granular polymer is easy to obtain. The gradual cooling preferably cools the liquid phase by controlling at a cooling rate of 2.0 to 0.1° C./minute. The gradual cooling can be performed by a method of exposing the polymerization reaction system to an ambient environment temperature (for example, room temperature). A method of flowing a coolant in a jacket of the polymerization reaction tank, or refluxing the liquid phase by a reflux condenser can be used in order to control the cooling rate of the liquid phase.

In the manufacturing method of the present invention, the liquid phase is preferably gradually cooled by controlling a cooling rate at preferably 2.0 to 0.1° C./minute, more preferably 1.5 to 0.2° C./minute, and even more preferably 1.3 to 0.3° C./minute until the temperature of the liquid phase in the polymerization reaction system reaches a polymerization temperature of the liquid-liquid phase separation polymerizing step, or until reduced from the liquid-liquid phase separation condition to 220° C. Granulation of the polymer can be promoted by controlling the cooling rate. The liquid phase can be cooled to a desire temperature without controlling the temperature from 220° C. From 220° C., the polymerization reaction system can be placed in an ambient environment temperature, or the cooling rate of the liquid phase can be increased. The final cooling temperature is a temperature that is room temperature to 220° C. where a separating/recovering step such as sieving or the like is easy. Preferably, the temperature is preferably 35° C. or higher, more preferably 40° C. or higher, and even more preferably 45° C. or higher in order to obtain a granular PAS with favorable thermal stability, low gas occurrences during a molding process, having low halogen content and low melt viscosity, and with a higher balanced performance. By setting the upper limit to preferably 200° C. or lower, and more preferably lower than 100° C., a slurry containing sufficiently granulated PAS can be obtained.

### 2-5. Separating/Recovering Step

With the manufacturing method of the present invention, granular PAS can be generated, and therefore, a method of separating and recovering the granular PAS from a reaction solution by a method of sieving using a sieve with a predetermined sieve opening can be used. Sieving may be performed while the product slurry is in a high temperature condition (for example, a temperature that is room temperature to 220° C.). In the manufacturing method of the present invention, sieving of the generated PAS is performed by a sieve having a 38 μm or greater sieve opening, and the PAS is recovered as the material remaining on the sieve after sieving. Sieving may be performed after washing described later, or after drying. Furthermore, sieving may be performed in stages before washing, after washing, and after drying.

Next, washing and filtering are repeated in accordance with a normal method. For example, the PAS is preferably washed by the same organic amide solvent as the polymerization solvent or ketones, (such as acetones), alcohols (such as methanols), or other organic solvent. The PAS may be washed by hot water or the like. The generated PAS can be treated by an organic acid or salt such as ammonium chloride. Acetic acid is preferably used as the organic acid. After washing, drying is performed in accordance with a normal method.

Specifically, a sieve with a 150 μm sieve opening (100 mesh (number of openings/inch), a sieve with a 105 μm sieve opening (145 mesh (number of openings/inch), a sieve with a 75 μm sieve opening (200 mesh (number of openings/inch), a sieve with a 38 μm sieve opening (400 mesh (number of openings/inch), or the like can be used as the sieve used for recovering the granular PAS, and therefore, a low molecular weight material or oligomer can be efficiently removed. A sieve with a 150 μm sieve opening (100 mesh (number of openings/inch) that can efficiently remove fine particulate byproduct salt is more preferably used.

With the manufacturing method of the present invention, the granular PAS collected in the sieve having a 150 μm sieve opening can be recovered at a yield rate that is normally 86.5 mass % or higher, preferably 87.5 mass % or higher, more preferably 88.5 mass % or higher, and even more preferably 89.0 mass % or higher.

### 3. Granular Polyarylene Sulfide

Granular PAS according to one embodiment of the present invention has a content of material on a sieve using a screen with a 150 μm opening diameter that is 86.5 mass % or greater, and a particle strength that is 88% or higher. Granular PAS according to another embodiment of the present invention has a specific surface area of 85 m²/g or less, and a particle strength of 88% or higher. Granular PAS according to yet another embodiment of the present invention has a yield retention rate of 76% or higher. The granular PAS can be manufactured by the method of manufacturing granular PAS according to the present invention for example.

Granular PAS according yet another embodiment of the present invention is manufactured by the method of manufacturing granular PAS according to the present invention. The granular PAS manufactured as described above has a content of material on a sieve using a screen with a 150 μm opening diameter that is 86.5 mass % or greater, and a particle strength that is 88% or higher. Furthermore, the granular PAS manufactured as described above has a specific surface area of 85 m²/g or less, and a particle strength of 88% or higher. Furthermore, the granular PAS manufactured as described above has a yield retention rate of 76% or higher.

Therefore, the granular PAS according to the present invention is excellent from the perspective of yield, particle strength, and/or yield retention rate.

The granular PAS according to the present invention has a content of material on a sieve using a screen with a 150 μm opening diameter of 86.5 mass % or higher, preferably 87.5 mass % or higher, more preferably 88.5 mass % or higher, and even more preferably 89.0 mass % or higher.

The granular PAS according to the present invention has a specific surface area of 85 m²/g or less, preferably 82 m²/g or less, more preferably 80 m²/g or less, and even more preferably 75 m²/g or less. Note that in the present specification, the specific surface area is measured by a BET method based on nitrogen adsorption.

The granular PAS according to the present invention has a particle strength of 88% or higher, preferably 89% or higher, more preferably 90% or higher, and even more preferably 93% or higher. Note that in the present specification, after shaking a mixture of 500 g of glass beads and 30 g of material on a 100 mesh sieve of the granular PAS for 30 minutes using a shaker, when the granular PAS is sieved by a 100 mesh screen, the particle strength is a mass ratio calculated by (mass of material on a 100 mesh sieve)/(total mass of material on a 100 mesh sieve and material that passed through a 100 mesh sieve)×100.

The granular PAS according to the present invention has a yield retention rate of 76% or higher (for example, 76% to 100%), preferably 77% to 100%, more preferably 78% to 100%, and even more preferably 79% to 100%. Note that in the present specification, the yield retention rate is defined by yield×particle strength×10⁻².

Of these, granular PAS manufactured by the method of manufacturing granular PAS according to embodiment A, having a weight average molecular weight of 60000 or less, preferably 15000 to 50000, more preferably 17000 to 48000, even more preferably 18000 to 45000, and yet even more preferably 20000 to 40000, having a content of material one a sieve using a screen with a 150 μm opening diameter that is 86.5 mass % or greater, preferably 87.5 mass % or greater, more preferably 88.5 mass % or greater, and, even more preferably 89.0 mass % or greater, and having a particle strength of 88% or higher, preferably 89% or higher, more preferably 90% or higher, and even more preferably 93% or higher is preferable from the perspective of the yield and particle strength of the granular PAS.

Of these, granular PAS manufactured by the method of manufacturing granular PAS according to embodiment B, having a specific surface area of 85 m²/g or less, preferably 82 m²/g or less, more preferably 80 m²/g, and even more preferably 75 m²/g or less, and having a particle strength of 88% or higher, preferably 89% or higher, more preferably 90% or higher, and even more preferably 93% or higher is preferable from the perspective of the particle strength of the granular PAS.

The granular PAS of the present invention can be molded as is or after oxidative crosslinking, into various injection molded products or extrusion molded products such as sheets, films, fibers, pipes, or the like independently or by adding various synthetic resins, various fillers, and various additives. The granular PAS is useful as a sealing agent or coating agent of an electronic component. The PAS is particularly preferably PPS.

### 4. Method of Improving Yield of Granular PAS

The present invention is a method of improving the yield of material on a sieve using a screen with a 150 μm opening diameter of granular PAS, including:
a polymerizing step of subjecting a dihalo aromatic compound and at least one type of sulfur source selected from the group consisting of alkali metal sulfides and alkali metal hydrosulfides to polymerization reaction in an organic amide solvent; and
a cooling step of cooling the reaction product mixture after the polymerizing step;
   where
a phase separation agent is added to the reaction product mixture at a point in time from the start of the polymerizing step to before the start of forming granular PAS in the cooling step.

The polymerizing step can provide a method including:
(1) a step of reacting a dihalo aromatic compound with an alkali meta sulfide at a temperature of 170 to 270° C., in an organic amide solvent containing from 0.5 to 2.4 mol of water per 1 mol of charged alkali metal sulfide, and setting the conversion ratio of the dihalo aromatic compound to 50 to 98 mol% to generate a prepolymer of a polyarlyene sulfide; and
(2) a step of adding water to a reaction system such that from 2.5 to 10 mol of water per 1 mol of charged alkali metal sulfide is in a present condition, continuing the reaction for 0.5 to 20 hours at a temperature of 245 to 290° C., increasing the conversion ratio of the dihalo aromatic compound, and converting the prepolymer to a polyarylene sulfide with a higher molecular weight. A detail description of the aforementioned yield improving method of the polymerizing step, cooling step, and the like are as described above.

In the yield improving method according to the present invention, the phase separation agent is added to the reaction product mixture at a point in time from the start of the polymerizing step to before the start of forming the granular PAS in the cooling step, and therefore, PAS with a moderate molecular weight in a melted condition has reduced solubility in a produced polymer dilute phase and transitions to a produced polymer dense phase with a high concentration of PAS with a high molecular weight. In this condition, granular PAS is formed in the cooling step, and therefore, PAS with a moderate molecular weight can be introduced in the granular PAS and recovered as a product. As a result, in the granular PAS, the yield of material on a sieve using a screen with a 150 μm opening diameter is improved.

### 5. Method of Improving Particle Strength of Granular PAS

The present invention can provide a particle strength improving method of granular PAS. In one embodiment, the particle strength improving method further includes:
a polymerizing step of subjecting a dihalo aromatic compound and at least one type of sulfur source selected from the group consisting of alkali metal sulfides and alkali metal hydrosulfides to polymerization reaction in an organic amide solvent; and
a cooling step of cooling the reaction product mixture after the polymerizing step;
   wherein
a specific surface area reducing step of reducing a specific surface area of the granular polyarylene sulfide is further included at a point in time from the start of the polyermizing step to before the start of forming a granular polyarylene sulfide in the cooling step.

In the present embodiment, by further including the specific surface area reducing step, the particle strength of the granular PAS can be improved in the same manner as described above.

In another embodiment, a particle strength improving method according to the present invention includes:
a polymerizing step of subjecting a dihalo aromatic compound and at least one type of sulfur source selected from the group consisting of alkali metal sulfides and alkali metal hydrosulfides to polymerization reaction in an organic amide solvent; and
a cooling step of cooling the reaction product mixture after the polymerizing step;
   where
addition of a phase separation agent to the reaction product mixture is started at a point in time from the start of the polymerizing step to before the start of forming granular PAS in the cooling step, and
when the temperature of the reaction product mixture is 245° C. or higher, 50 mass% or greater of the phase separation agent is added to the reaction product mixture; the polymerizing step, including:
(1) a step of reacting a dihalo aromatic compound with an alkali meta sulfide at a temperature of 170 to 270° C., in an organic amide solvent containing from 0.5 to 2.4 mol of water per 1 mol of charged alkali metal sulfide, and setting the conversion ratio of the dihalo aromatic compound to 50 to 98 mol % to generate a prepolymer of a polyarlyene sulfide; and
(2) a step of adding water to a reaction system such that from 2.5 to 10 mol of water per 1 mol of charged alkali metal sulfide is in a present condition, continuing the reaction for 0.5 to 20 hours at a temperature of 245 to 290° C., increasing the conversion ratio of the dihalo aromatic compound, and converting the prepolymer to a polyarylene sulfide with a higher molecular weight.

In the present embodiment, addition of the phase separation agent to the reaction product mixture is started at a point in time from the start of a polymerizing step to before the start of forming granular PAS in the cooling step, and when the temperature of the reaction product mixture is 245° C. or higher, 50 mass % or greater of the phase separation agent is added to the reaction product mixture, and therefore, the particle strength of the granular PAS can be improved in the same manner as described above.

A detailed description of the polymerizing step, cooling step, specific surface area reducing step, and the like are described above for all of the aforementioned embodiments.

### EXAMPLES

### (1) Yield of Granular PAS

For the yield (mass %) of the granular PAS, the total amount of the PAS before sieving and the amount of material on a sieve or material that passed through a sieve collected by a sieve with a 38 μm sieve opening (400 mesh (number of openings/inch) or 150 μm sieve opening (100 mesh (number of openings/inch) and then calculated by (material on a sieve or material passed through a sieve/total amount)×100. The results are shown in Table 1.

### (2) Method Measuring Melt Viscosity

The melt viscosity of the granular PAS was measured by a Capillograph 1C (trade name) available from Toyo Seiki Seisaku-sho, Ltd. equipped with a 1.0 mm diameter, 10.0 mm long nozzle as a capillary. The set temperature was 310° C. A polymer sample was introduced in the device, the sample was maintained for 5 minutes, and then the melt viscosity was measured at a shear rate of 1200 sec⁻¹.

### (3) Average Particle Size

The average particle size of the granular PAS recovered in the separating/recovering step was measured by a sieving method using sieves with a 2800 μm sieve opening (7 mesh (number of openings/inch), 1410 μm sieve opening (12 mesh (number of openings/inch), 1000 μm sieve opening (16 mesh (number of openings/inch), 710 μm sieve opening (24 mesh (number of openings/inch), 500 μm sieve opening (32 mesh (number of openings/inch), 250 μm sieve opening (60 mesh (number of openings/inch), 150 μm sieve opening (100 mesh (number of openings/inch), 105 μm sieve opening (145 mesh (number of openings/inch), 75 μm sieve opening (200 mesh (number of openings/inch), and 38 μm sieve opening (400 mesh (number of openings/inch) as the used sieves, and then the average particle size when the cumulative mass reached 50% mass was calculated from the mass of material on the sieves. The results are shown in Table 1.

### (4) Specific Surface Area

The specific surface area of the granular PAS was measured by a BET method based on nitrogen adsorption, using a FlowSorb II 2300 available from Shimadzu Corporation. Prior to measurements, granular PPS on a 100 mesh sieve was washed with acetone three times and washed with water five times, and then granular PPS vacuum dried for 13 hours at 60° C. was sieved by a sieve with a 500 μm sieve opening (32 mesh (number of openings/inch)), and material that passed through the sieve with a 500 μm sieve opening (32 mesh (number of openings/inch)) was further sieved by a sieve with a 350 μm sieve opening (45 mesh (number of openings/inch)). Approximately 0.1 g of material on the sieve with a 350 μm sieve opening (45 mesh (number of openings/inch)) was used in the measurements.

### (5) Particle Strength

500 g of glass beads and 30 g of material on a 100 mesh sieve of granular PAS were inserted in a 1 L polyethylene bottle, and then shaken for 30 minutes by a shaker. After shaking, granular PAS inside the polyethylene bottle was sieved by a 100 mesh screen, and then the mass ratio of material on the 100 mesh sieve with regard to the total amount of the material on the 100 mesh sieve and material that passed through the 100 mesh sieve is determined and set as the particle strength. The results are shown in Table 1.

### (6) Weight Average Molecular Weight

The weight average molecular weight (Mw) of the polymer was measured under the following conditions, using a high temperature gel permeation chromatograph (GPC) SSC-7000 available from Senshu Scientific, Co., Ltd. The weight average molecular weight was calculated as a value based on polystyrene.
Solvent: 1-chloronaphthalene,
Temperature: 210° C.
Detector: UV detector (360 nm),
Sample injection amount: 200 μL (concentration: 0.05 mass %)
Flow rate: 0.7 mL/min
Standard polystyrene: five types of standard polystyrenes of 616,000, 113,000, 26,000, 8,200, and 600

### Example 1

### Dehydrating Step

5998 g of N-methyl-2-pyrrolidone (NMP), 2004 g of a sodium hydrosulfide aqueous solution (NaSH: purity 62.5 mass %), and 1205 g of sodium hydroxide (NaOH: purity 73.5 mass %) were introduced into a 20 L Ti-line autoclave.

After purging the inside of the autoclave with nitrogen gas, the temperature was gradually increased to 200° C. while stirring at a stirrer rotational speed of 250 rpm over 2.5 hours, and then 981 g of water H₂O), 921 g of NMP, and 11 g of hydrogen sulfide were distilled.

### Polymerizing Step

After the dehydrating step, the contents of the autoclave were cooled to 170° C., and 3400 g of pDCB, 3354 g of NMP, 20.9 g of sodium hydroxide (NaOH: purity 97.0 mass %), and 107 g of water were added, and the temperature was continuously increased to 260° C. over 3 hours while stirring to perform first-stage polymerization. The ratio (g/mol) of NMP/added sulfur source (hereinafter abbreviated as “added S”) in the can was 383, pDCB/added S (mol/mol) was 1.050, and H₂O/added S (mol/mol) was 1.50.

The pDCB conversion ratio during the first-stage polymerization step was 92%. After completing the first-stage polymerization step, the rotational speed of the stirrer was increased to 400 rpm, and 449 g of water was added while stirring the contents of the autoclave. H₂O/added S (mol/mol) was 2.63. After injecting water, the temperature was increased to 265° C., and the second-stage polymerization was performed by reacting for 2 hours.

After completing the second-stage polymerization, 79 g of water was started to be injected at 265° C. while cooling at a cooling rate of 0.8 to 1.0° C./min, and the injection was ended at 264° C. H₂O/added S (mol/mol) during cooling was 2.83.

### Separating Step

After further cooling at a cooling rate of 0.8 to 1.0° C./min to approximately room temperature, the contents were sieved by a screen with a 150 μm (100 mesh) and 38 μm (400 mesh) opening diameter a wet cake of granular PPS (preferably granulated body) was obtained on the sieve of the screen with a 150 μm opening diameter, a wet cake of fine granular PPS (38 μm to 150 μm fine powder) was obtained on the sieve of the screen with a 38 μm opening diameter, and a separated liquid (very fine powder where the solid contents passed through 38 μm sieve) was obtained below the sieve. Thereafter, the granular PPS on the 100 mesh sieve was washed with acetone three times and then washed with water five times to obtain granular PPS at yield of 89.3 mass %. The melt viscosity of the obtained PPS was 24 Pa·s, the average particle size was 480 μm, and the specific surface area was 70 m²/g. The particle strength was 94%. The yield retention rate as defined by yield×particle strength×10⁻² was 83.9%.

### Example 2

Other than starting water injection at 260° C., and ending injection at 255° C. after completing the second-stage polymerization in Example 1, all steps were performed similarly to Example 1. Granular PPS was obtained at a yield of 87.6 mass %. The melt viscosity of the obtained PPS was 25 Pa·s, the average particle size was 390 μm, and the specific surface area was 80 m²/g. The particle strength was 92%. The yield retention rate was 80.6%.

### Example 3

Other than starting water injection at 260° C., and ending injection at 248° C. after completing the second-stage polymerization in Example 1, all steps were performed similarly to Example 1. Granular PPS as a product was obtained at a yield of 87.7 mass %. The melt viscosity of the obtained PPS was 24 Pa·s, the average particle size was 430 μm, and the specific surface area was 81 m²/g. The particle strength was 89%. The yield retention rate was 78.1%.

### Comparative Example 1

Other than not adding water after completing the second-stage polymerization in Example 1, all steps were performed similarly to Example 1. Granular PPS was obtained at a yield of 86.1 mass %. The melt viscosity of the obtained PPS was 25 Pa·s, the average particle size was 460 μm, and the specific surface area was 86 m²/g. The particle strength was 87%. The yield retention rate was 74.9%.

TABLE 1

| | | | | | Compar- |
|---|---|---|---|---|---|
| | | Exam- | Exam- | Exam- | ative |
| | | ples | ples | ples | exam- |
| | | 1 | 2 | 3 | ples 1 |
| On sieve with 150 μm | mass | 89.3 | 87.6 | 87.7 | 86.1 |
| sieve opening | % | | | | |
| Passed through sieve | mass | 3.2 | 3.6 | 5.2 | 4.6 |
| with 150 μm sieve | % | | | | |
| opening | | | | | |
| On sieve with | | | | | |
| 38 μm sieve opening | | | | | |
| Passed through sieve | mass | 7.5 | 8.8 | 7.1 | 9.3 |
| with 38 μm sieve | % | | | | |
| opening | | | | | |
| Average particle size | μm | 480 | 390 | 430 | 460 |
| Specific surface area | m²/g | 70 | 80 | 81 | 86 |
| Particle strength | % | 94 | 92 | 89 | 87 |
| Yield retention rate | % | 83.9 | 80.6 | 78.1 | 74.9 |

1. A method of manufacturing granular polyarylene sulfide, the method comprising:
a polymerizing step of subjecting a dihalo aromatic compound and at least one type of sulfur source selected from the group consisting of alkali metal sulfides and alkali metal hydrosulfides to polymerization reaction in an organic amide solvent; and
a cooling step of cooling the reaction product mixture after the polymerizing step;
wherein
addition of a phase separation agent to the reaction product mixture is started at a point in time from after ending the polymerizing step to before the start of forming granular polyarylene sulfide in the cooling step, and
when the temperature of the reaction product mixture is 245° C. or higher, 50 mass % or greater of the phase separation agent is added to the reaction product mixture;
the polymerizing step comprising:
(1) a step of reacting a dihalo aromatic compound with an alkali meta sulfide at a temperature of 170 to 270° C., in an organic amide solvent containing from 0.5 to 2.4 mol of water per 1 mol of charged alkali metal sulfide, and setting the conversion ratio of the dihalo aromatic compound to 50 to 98 mol % to generate a prepolymer of a polyarlyene sulfide; and
(2) a step of adding water to a reaction system such that from 2.5 to 10 mol of water per 1 mol of charged alkali metal sulfide is in a present condition, continuing the reaction for 0.5 to 20 hours at a temperature of 245 to 290° C., increasing the conversion ratio of the dihalo aromatic compound, and converting the prepolymer to a polyarylene sulfide with a higher molecular weight.

2. (canceled)

3. The method of manufacturing granular polyarylene sulfide according to claim 1, wherein the phase separation agent is a particle modifier having a function of improving particle properties of the granular polyarylene sulfide.

4. The method of manufacturing granular polyarylene sulfide according to claim 1, wherein the polymerization temperature in the polymerizing step is 250° C. or higher.

5. Granular polyarylene sulfide with a weight average molecular weight of 60000 or less, manufactured by the method of manufacturing granular polyarylene sulfide according to claim 1, wherein an amount of product sieved by a screen with a 150 μm opening diameter is 86.5 mass % or greater, and the granular polyarylene sulfide has a particle strength of 88% or higher.

6. (canceled)

7. (canceled)
